(19) United States
(12) Patent Application Publication
(10) Pub. No.: US 2006/0140885 A1
(43) Pub. Date: Jun. 29, 2006

(54) Method of reducing oral tissue inflammation using magnolia extract

(76) Inventors: Abdul Gaffar, Princeton, NJ (US); Dale Scherl, Lawrence, KS (US); Susan Herles, Flemington, NJ (US)

Correspondence Address: COLGATE-PALMOLIVE COMPANY, 909 RIVER ROAD, PISCATAWAY, NJ, 08855, US

(21) Appl. No.: 11/285,809

(22) Filed: Nov. 23, 2005

Related U.S. Application Data
(60) Provisional application No. 60/640,161, filed on Dec. 29, 2004.

(51) Int. Cl. A61K 8/97; A61K 36/575

(52) U.S. Cl. 424/58; 424/769

(57) ABSTRACT

A method for treating a mammal having oral tissue inflammation is provided, where the inflamed oral tissue is contacted with a safe, efficacious, non-irritating oral composition having an anti-inflammatory agent comprising a magnolia extract. The magnolia anti-inflammatory active ingredient reduces one or more mediators of inflammation and reduces inflammation in oral tissue. The oral composition can be in the form of a mouth rinse; dentifrice, including toothpaste, gels, powders, lozenges; medicament gel; animal products; and the like.

### CROSS REFERENCE TO RELATED APPLICATIONS

This application claims priority to U.S. Provisional Patent Application Ser. No. 60/640,161, filed Dec. 29, 2004, the contents of which are incorporated herein by reference.

### BACKGROUND OF THE INVENTION

Gingivitis is the inflammation or infection of the gums and the alveolar bones that support the teeth. Gingivitis is generally believed to be caused by bacteria in the mouth (particularly the bacteria instigated in plaque formation) and the toxins formed as by-products from the bacteria. The toxins are believed to instigate oral tissue inflammation within the mouth. Periodontitis is a progressively worsened state of disease as compared to gingivitis, where the gums are inflamed and begin to recede from the teeth and pockets form, which ultimately may result in destruction of the bone and periodontal ligament. Bacterial infections of the structures that support the dentition can include gingivitis and periodontitis, but may also include infections of the bone, for example the mandibles as a result of surgical intervention. Further, oral tissue inflammation can be caused by surgery, localized injury, trauma, necrosis, improper oral hygiene or various systemic origins.

It is generally believed that the cellular components implicated by these diseases and conditions include epithelial tissue, gingival fibroblasts, and circulating leukocytes, all of which contribute to the host response to pathogenic factors generated by the bacteria. The most common bacterial pathogens implicated in these oral infections are Streptococci spp. (e.g., S. mutans), Porphyromonas spp., Actinobacillus spp., Bacteroides spp., and Staphylococci spp., Fusobacterium nucleatum, Veillonella parvula, Actinomyces naeslundii, and Porphyromonas gingivalis. Although the bacterial infection is often the etiological event in many of these oral diseases, the pathogenesis of the disease is mediated by the host response. Circulating polymorphonuclear neutrophils (PMNs) are largely responsible for the hyperactivity found at sites of infection. Typically PMNs and other cellular mediators of inflammation become hyper-functional and release toxic chemicals that are partly responsible for the destruction of tissue surrounding the foci of infection.

Thus, bacterial infection of the oral tissue stimulates the host's immune response and diminishes the healing process by up-regulating inflammatory mediators that cause significant tissue damage. One class of mediators extensively studied for their effect on the inflammatory response is the arachidonic acid metabolites namely prostaglandins and leukotrienes, that are produced through the cyclooxygenase or lipoxygenase enzyme pathways. These metabolites have been implicated as the prime mediators in gingivitis, periodontitis, osteomyelitis and other inflammatory diseases.

There are a variety of compositions described in the art for preventing and treating oral inflammation as a result of bacterial infection. In particular, to prevent the accumulation of inflammatory mediators derived from arachidonic acid pathway, non-steroidal anti-inflammatory drugs (NSAIDs) have been used successfully to treat patients suffering from periodontal disease and inflammatory diseases that are caused by arachidonic acid metabolites. Experimental and clinical data have shown that indomethacin, flurbiprofen, ketoprofen, ibuprofen, naproxen, and meclofenamic acid have significant ameliorative effects against alveolar bone loss, and reduction of prostaglandins and leukotrienes in dental disease models. However, one major disadvantage to the regular use of NSAIDs is the potential development of heartburn, gastric ulcers, gastrointestinal bleeding, and toxicity.

Other treatment methods include the use of antimicrobial therapeutics and antibiotics to eliminate the underlying infection. These treatments operate to reduce the source of irritants (bacteria), but are slow to affect the host immune response to the toxins secreted by the bacteria. In addition, certain antibiotics and other antimicrobial therapeutics potentially cause ulceration of oral mucous membranes, induction of desquamative gingivitis, discoloration, the potential for antibiotic resistance after prolonged usage, as well as exacerbation of tissue inflammation due to irritation. There is a need for a non-irritating anti-inflammatory oral composition that can effectively reduce oral tissue inflammation in progressively diseased mammalian subjects.

### BRIEF SUMMARY OF THE INVENTION

In one embodiment, a method of treating a mammalian subject having oral tissue inflammation is provided. The method comprises contacting the inflamed oral tissue with an oral composition comprising an anti-inflammatory active ingredient consisting essentially of an extract of magnolia and an orally acceptable carrier. The oral composition reduces inflammation of the oral tissue by reducing one or more mediators of inflammation.

In another embodiment, a method is provided for reducing oral tissue inflammation in a mammalian subject. The inflamed oral tissue is contacted with an oral composition comprising a non-irritating amount of an anti-inflammatory active ingredient consisting essentially of an extract of magnolia, and an orally acceptable carrier. The oral composition does not irritate the inflamed oral tissue, and further reduces inflammation of the oral tissue by reducing one or more mediators of inflammation.

In an embodiment of the present invention, a method for treating a mammalian subject having oral tissue inflammation is provided. The method comprises contacting the inflamed oral tissue with an oral composition comprising an anti-inflammatory active ingredient consisting essentially of an extract of magnolia and an orally acceptable carrier. The oral composition reduces inflammation by reducing one or more mediators of inflammation. The orally acceptable carrier comprises one or more oral active ingredients selected from the group consisting of: anti-tartar agents, antibacterial agents, anticaries agents, whitening agents, densensitizing agents, vitamins, compatible enzymes, breath freshening agents, malodor preventing agents, and combinations thereof.

It has been discovered that compositions and methods of this invention impart advantages over the prior art oral anti-inflammatory compositions, by providing an oral care composition that is safe, stable, non-irritating, and highly effective as an anti-inflammatory and analgesic treatment. Further, the oral composition comprises an anti-inflammatory constituent that is natural and derived from a botanical source. Further uses, benefits and embodiments of the present invention are apparent from the description set forth herein.

### DETAILED DESCRIPTION OF THE INVENTION

The present invention provides a method of treating a mammalian subject having oral tissue inflammation in an oral cavity. The method comprises contacting an oral composition comprising a safe, effective, and non-irritating.

“Inflammation” of the oral tissue generally refers to a localized protective response elicited by injury or destruction of tissues, which serves to destroy, dilute, or sequester both the injurious agent and the injured tissue. In the acute form, it is characterized by pain, heat, redness, swelling, and loss of function. Chronic inflammation is a slow process and primarily characterized by the formation of new connective tissue. Chronic inflammation is often a continuation of acute inflammation or a prolonged low-grade form of inflammation (such as that associated with periodontitis or gingivitis) and usually causes permanent tissue damage. Histologically, inflammation involves a complex series of events, including dilation of arterioles, capillaries, and venules, with increased permeability and blood flow; exudation of fluids, including plasma proteins, and leukocytic migration into the inflammatory locus. Inflammation corresponds to enhanced levels of pro-inflammatory cellular mediators, or substances that are released from cells, for example, as the result of the interaction of an antigen with an antibody or by the action of antigen with a sensitized lymphocyte.

In certain embodiments, when the oral composition is contacted with the oral tissue, it provides an analgesic effect on the inflamed oral tissue, thereby reducing sensations of pain and sensitivity in the oral tissue in the mammalian subject. In certain embodiments, the contacting of the oral care composition to the inflamed oral tissue is repeated at regular intervals.

Thus, in various embodiments of the present invention, the oral composition comprising magnolia is applied to sites of inflamed oral tissue at a concentration that reduces the production of one or more inflammatory cellular mediators. In various embodiments of the present invention, the anti-inflammatory magnolia active ingredient of the oral composition simultaneously inhibits formation of multiple proinflammatory mediators, for example, both PGE₂ and TNF-α Each respective mediator generally has a different mechanism in the pathogenesis of a disease.

Thus, in certain embodiments of the present invention, the oral composition comprising an anti-inflammatory ingredient comprising magnolia can further function to offset the innate effects of bone resorption and inhibition of bone formation as a result of over production and activity of cellular mediator molecules, such as PGE₂ and TNF-α. In this manner, certain embodiments of the present invention provide methods for reducing alveolar bone loss, tooth loss and damage to mandibular bone as a result of trauma and/or infection in patients experiencing inflammation by applying various embodiments of the oral composition of the present invention comprising magnolia extract directly to the affected inflamed oral tissue surface.

In various embodiments, the oral compositions comprise an anti-inflammatory agent at a concentration where the production of one or more proinflammatory mediators, such as for example, PGE₂ or TNF-α is significantly diminished. However, as recognized by one of skill in the art, a complete suppression of formation of such cellular mediators is also potentially detrimental to the mammalian subject, and in accordance with certain embodiments of the present invention, the production of cytokines is not entirely repressed. Thus, in various embodiments, the magnolia extract active ingredient is present in the oral composition at a concentration that prevents the over-expression of one or more inflammatory mediators (which prevents an intrinsic mechanism for chronic disease), but still permits sufficient production of certain desirable mediator molecules (which are pleiotropic) to maintain homeostasis and normal cellular functions at basal levels.

Sources of oral tissue inflammation include bacterial infection, surgery, localized injury, trauma or necrosis, various systemic origins, or non-disease related etiologies such as overly aggressive oral hygiene practices or inappropriate dental hygiene practices. Non limiting examples of oral diseases, conditions, and disorders associated with enhanced activity of cellular mediators of inflammation include gingivitis, periodontitis, stomatitis, exfoliation of teeth due to neutropenia, endodontic pathoses and its sequela, acute and chronic ulceration of the oral mucosa, acute necrotizing ulcerative gingivitis, osteoclast/ondontoclast mediated resorptive legions, dental caries, delayed wound healing, periodontal bone damage and acute and chronic osteomyelitis of the mandibular bone.

In certain embodiments, the present invention is useful for preventing the development of diseases. As used herein the term “prevention” pertains to a prophylactic treatment of an oral cavity of a mammalian subject, by contacting an oral composition comprising an anti-inflammatory active ingredient with oral tissue having a propensity for becoming inflamed, diseased, or damaged.

In certain embodiments, a method is provided for treating diseases and disorders of the oral cavity and conditions associated with inflammation, infection and elevated levels of one or more pro-inflammatory cellular mediators in the oral cavity. “Treating” involves the application of an oral composition comprising the magnolia extract after the development or physical manifestation of inflammatory response due to a disease or condition. Upon treating the inflamed tissue, the inflammation, disease, or condition is ameliorated or prevented from deteriorating to a worsened state. For example, the application of magnolia extract after the development of the inflammatory cascade comprises “treatment” of the disease or inflammatory/infectious symptoms.

In certain embodiments, the method of treatment comprises administering a therapeutically beneficial amount of magnolia extract at repeated intervals over a period time, from one week up to a lifetime. For example, a typical method for treating diseases, conditions, and disorders of the oral cavity that are associated with inflammation, infection and elevated levels of one or more inflammation mediators comprises administration of a therapeutically beneficial amount of an oral composition comprising magnolia extract, administered on a daily basis.

In various embodiments, application or contacting can be accomplished by rinsing, coating, brushing, or layering using appropriate dressing materials. Further, contacting can include incidental contact during eating or chewing. In various embodiments, application of the composition comprises the use of an application device which aids in maintaining the contact time of the anti-inflammatory active ingredient comprising magnolia extract to the target tissue for a sufficient time as to allow the pharmacological inhibition of the elevated production of one or more inflammatory mediators, such as PGE₂ and TNF-α.

The present invention provides a highly effective oral composition for reducing inflammation of oral tissue in a mammalian subject. The oral composition comprises an anti-inflammatory ingredient consisting essentially of an extract of magnolia and an orally acceptable carrier.

As referred to herein, an “oral care composition” is any composition that is suitable for administration or application to the oral cavity and surrounding oral tissues of a mammalian subject. In various embodiments, an oral care composition is not intentionally swallowed, but rather is retained in the oral cavity for a time sufficient to effect the intended utility. In certain embodiments, particularly those where the oral composition is provided in an animal product, such as a pet food, pet food supplement (e.g., a treat), or a chew toy, the oral composition may be ingested at small concentrations which are not harmful to the animal. Preferably, specific materials and compositions to be used in this invention are pharmaceutically- or cosmetically-acceptable. As used herein, such an “orally acceptable” or “cosmetically acceptable” component is one that is suitable for use with humans and/or animals to provide the desired therapeutic, prophylactic, sensory, decorative, or cosmetic benefit without undue adverse side effects (such as toxicity, irritation, and allergic response) commensurate with a reasonable benefit/risk ratio. The present invention provides methods of use to provide therapeutic benefits using oral care compositions having an active ingredient comprising an extract of magnolia for use with a human or other mammalian animal subject that has inflamed oral tissue.

The compositions of the present invention comprise an extract of magnolia. As referred to here, such an “extract” of magnolia is an extract from dried cortex, or bark, of a plant from the Magnoliaceae family, such as Magnolia officinalis, (hereinafter “magnolia”) or a synthetic or semi-synthetic equivalent of such an extract or an active component or compound thereof. Preferably, extracts of Magnolia Cortex (the bark of Magnolia officinalis) contain active compounds including: magnolol, honokiol, tetrahydromagnolol, and tetrahydrohonokiol, which have demonstrated bactericidal properties against S. mutans in the in vitro test Minimal Inhibitory Concentration (MIC). It should be noted that any plant from the Magnoliaceae family is suitable for the present invention and may be used in alternate embodiments, preferably such that the extract comprises an antimicrobially effective concentration of a compound selected from the group consisting of magnolol, honokiol, tetrahydromagnolol, tetrahydrohonokiol, and mixtures thereof.

Magnolia extract reduces the expression of one or more proinflammatory mediators in oral tissue, particularly cytokines, including prostaglandins, leukotrienes, tumor necrosis factor-alpha (TNF-α), interleukins, and the inducible form of nitric oxide using cell culture in vitro experiments. Magnolia extract also reduces PMN infiltration to sites of challenge using animal models.

As used herein, “extracting” or “extraction” of a solid or liquid material means contacting the material with an appropriate solvent to remove the substance(s) desired to be extracted from the material. Where the material is solid, it is preferably dried and crushed or ground prior to contacting it with the solvent. Such an extraction may be carried out by conventional means known to one of skill in the art, for example, by using an extraction apparatus, such as a Soxhlet apparatus, which retains the solid material in a holder and allows the solvent to flow through the material; or by blending the solvent and material together and then separating the liquid and solid phases or two immiscible liquid phases, such as by filtration or by settling and decanting.

In one embodiment, Magnolia Extract is made from dried Magnolia plant bark and can be prepared by extracting the bark using an appropriate solvent. Preferred solvents include methanol, ethanol, methylene chloride, hexane cyclohexane, pentane, petroleum ether, chloroform, hydrochloric acid, ethylene dichloride, and hydrofluoroalkanes, such as 1,1,1,2-tetrafluoroethane or HFA-13A. Generally, one part of plant tissue (dry basis) is extracted with about 5 to about 50 parts, preferably about 15 parts to about 30 parts of solvent using an extraction apparatus where the solvent is contacted with the bark to obtain a concentrated paste which is then subjected to one or more additional extraction steps with different solvents to further concentrate the originally obtained paste over an extended period of time, preferably about 6 hours to about 1-2 days, more preferably for about 1 day. In one simplified method of extraction, the dried, crushed Magnolia bark in the form of a powder is contacted with a hydrofluoroalkane (such as, 1,1,1,2-tetrafluoroethane (HFA-13A)) to form a concentrated final extraction yielding an extract containing about 5 to about 50% honokiol and about 5 to about 50% magnolol.

In preferred embodiments, the natural extract active ingredients used in oral care compositions are of reproducible, stable, and have microbiological safety. In one embodiment of the present invention, the magnolia extract is isolated by supercritical fluid extraction (SFE) using carbon dioxide (CO₂). Supercritical fluids are gases with properties between that of a “normal” phase of gas and liquid. Pressure variations control the properties of the supercritical fluids, which can range from more gas-like behavior to more liquid-like behavior, depending on the application. Supercritical fluids use a solvent that is readily available, inexpensive, and environmentally safe (CO₂ and H₂O). Carbon dioxide is non-toxic, non-explosive, readily available and easily removed from the extracted products. Process temperatures for SFE are generally low to moderate. Thus, SFE produces nearly solvent-free products, and further avoid any potential deterioration reactions.

Natural contaminants that may be potentially present in other extraction methodologies are generally absent in the SFE extracted product. For example, compounds such as aristocholic acid and alkaloids, such as magnocurine and tubocurarine, are kept at low concentrations (for example, generally less than 0.0002 percent). Thus, in the embodiment where the magnolia is extracted by SFE, the extract is substantially free from chemical alterations brought about by heat and water, from solvent residues, and other artifacts.

Further, certain magnolia SFE extracts are very cosmetically acceptable. Certain methods of magnolia extraction produce a dark brown product that is difficult to formulate in an oral care composition, due to the dark color, even at low concentrations. In certain embodiments, SFE extraction produces a much lighter color of magnolia extract (a light beige product) that is particularly suitable for aesthetically pleasing oral composition formulations.

In various embodiments, it is preferred that the active antibacterial ingredient comprises either magnolol, honokiol, or both. Magnolol and honokiol are non-ionic hydroxybiphenyl compounds, the structures of which are believed to be as follows: 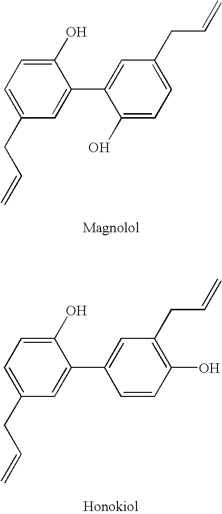
Additionally, tetrahydromagnolol and tetrahydrohonokiol are hydrogenated analogs of magnolol and honokiol often found in relatively small concentrations in the extracts of magnolia, and as such may be included in the anti-inflammatory ingredient.

Thus, as will be described in greater detail below, in various embodiments of the present invention, an effective amount of magnolia extract comprises one or more active compounds: magnolol, honokiol, tertrahydromagnolol and tetrahydrohonokiol and mixtures thereof, which are used to inhibit the excess production of cellular mediators of inflammation in oral tissue at sites of inflammation caused by infection, environmental toxins, or trauma in the oral cavity. An effective amount of magnolia extract reduces the levels or activity of proinflammatory mediators adequately to reduce the concentration in the mammalian subject to basal levels in the oral tissue of the subjects treated, without unnecessarily suppressing all intercellular mediator activity.

In various embodiments, the magnolia extract of the present invention comprises magnolol, honokiol, or both in an amount of about 2% to about 95%. In other embodiments, the magnolia extract comprises magnolol, honokiol, or both in an amount of about to about 50%. In one embodiment of the present invention, the magnolol is present in an amount of about 30 to 50%. In another embodiment, honokiol is present in an amount of about to 50%, more preferably in an amount of about 30 to 50%. Magnolia extracts among those useful herein are commercially available. One such extract is obtained by HFA-13A extraction and comprises magnolol at about 37% and honokiol at about 15%.

Additionally, the concentration of magnolia extract in the oral care composition depends upon the relative concentration of the active compounds in the extract, and as such, it is contemplated that the amount of magnolia extract present may vary as recognized by one of skill in the art. The concentration of the active ingredients is typically dependent upon the form of the oral composition. For example, mouthrinses typically have a relatively low concentration of an active ingredient, as where dentifrices, gels, or toothpowders have a higher concentration to achieve the same delivered dosage based on ease of dispersion. Likewise, confectionary compositions typically have a relatively wide range of concentrations of active ingredient to enable sufficient dispersion as they dissolve or are masticated.

While not limiting to theories by which the present invention is bound, it is generally believed that a bactericidal level (Minimum Inhibitory Concentration) of magnolia extract (as measured by magnolol, honokiol, or the combination of both active compounds) is between about 10 μg/mL (mg/kg or parts per million (ppm)) to about 20 μg/mL (ppm) near the targeted site within the oral cavity. For example, it is speculated that in some circumstances, a minimum inhibitory concentration (MIC) or a bactericidal level is approximately between about 8 μg/mL (ppm) to about 16 μg/mL (ppm) for residual active compounds in the oral cavity.

In highly sensitive tissue, high concentrations of magnolia may potentially cause irritation and exacerbate inflammation, rather than reduce it. While the potential for additional inflammation is dependent upon the individual subject's status and response to irritants, as well as other variables related to treatment, it is preferred that the magnolia extract is provided to the subject at a non-irritating concentration. By “non-irritating” it is meant that the contact of the oral composition with the active ingredient comprising magnolia extract does not increase soreness, pain, redness, or roughness, nor does it exacerbate or worsen inflammation of the oral tissue.

Thus, while it is beneficial for the magnolia extract to have both bactericidal and anti-inflammatory effects, in some circumstances, a non-irritating concentration that is anti-inflammatory may fall below the bactericidal concentration for magnolia. Further, at high concentrations magnolia has potential to discolor teeth. Thus, in some embodiments of the present invention, the magnolia extract has a relatively low targeted delivery dosage to the inflamed tissue, and can be assessed by the residual concentration of magnolia active compounds in pooled plaque samples an hour after application. For example, in certain embodiments, the concentration of magnolol and/or honokiol is less than about 20 μg/mL. In other embodiments, the magnolia extract present in the pooled plaque samples is less than about 10 μg/mL. In certain embodiments, the magnolia extract is present at a concentration of less than about 5 μg/mL in pooled plaque samples. At various concentrations, the magnolia extract has efficacy as an anti-inflammatory. In some embodiments, the anti-inflammatory magnolia active is delivered at a relatively low concentration that has both anti-inflammatory effects, as well as anti-bacterial effects. In other embodiments, the anti-inflammatory magnolia active provides only anti-inflammatory efficacy to the inflamed tissue because the dosage is less than a bactericidal level.

In other embodiments of the present invention, the magnolia extract is present in the oral care composition in an amount of about 0.001 to about 10%. As appreciated by one of skill in the art, such a concentration is dependent upon the concentration of active ingredients. In one embodiment, the magnolia extract is present in the oral care composition in an amount of about 0.001 to about 3%. In other embodiments, the magnolia extract is present at less than 1%, for example the extract is present at a concentration of in an amount of about 0.01 to about 1%. In one preferred embodiment, the magnolia extract is present in the oral care composition at a concentration of about 0.3%.

In various embodiments of the present invention, the oral composition comprises an anti-inflammatory ingredient consisting essentially of magnolia, and an orally acceptable carrier. As used herein, an “orally acceptable carrier” refers to a material or combination of materials that are safe for use in the compositions of the present invention, commensurate with a reasonable benefit/risk ratio, with which the magnolia extract may be associated while retaining significant efficacy. The orally acceptable carrier may comprise a variety of other conventional active ingredients known to one of skill in the art, including, tartar control agents, anticaries agents, sensitivity agents, and the like. Preferably, the carrier does not substantially reduce the efficacy of the anti-inflammatory active ingredient consisting essentially of magnolia extract.

A suitable vehicle or carrier includes one or more compatible solid or liquid fillers, diluents, excipients, or encapsulating substances which are suitable for topical administration to oral tissue surfaces. It is preferred that the orally acceptable carrier does not cause irritation, swelling or pain and does not typically produce an allergic or untoward reaction such as gastric upset, nausea or dizziness. Selection of specific carrier components is dependant on the desired product form, including dentifrices, toothpastes, tooth powders, prophylaxis pastes, mouth rinses, lozenges, gums, gels, paints, and the like.

In various embodiments, the orally acceptable dentifrice carrier used to prepare an oral composition comprises a water-phase. As recognized by one of skill in the art, the oral compositions of the present invention optionally include other materials, such as for example, viscosity modifiers, diluents, surface active agents, such as surfactants, emulsifiers, and foam modulators, pH modifying agents, abrasives, humectants, mouth feel agents, sweetening agents, flavor agents, colorants, preservatives and combinations thereof. It is understood that while general attributes of each of the above categories of materials may differ, there may be some common attributes and any given material may serve multiple purposes within two or more of such categories of materials. Preferably, such carrier materials are selected for compatibility with the anionic antibacterial magnolia active ingredient, as well as with other ingredients of the composition.

The term “mouthrinse” in the present invention refers to oral compositions that are substantially liquid in character, such as a mouth wash, spray, or rinse. In such a preparation the orally acceptable carrier typically has an aqueous phase comprising water or a water and alcohol mixture. Further, in various embodiments, the oral carrier has a humectant and surfactant as described below. Generally, the weight ratio of water to alcohol is in the range of in an amount of about 1:1 to about 20:1, preferably about 3:1 to 10:1 and more preferably about 4:1 to about 6:1. The total amount of water-alcohol mixture in this type of preparation is typically in an amount of about 70 to about 99.9% of the preparation. In various embodiments, the alcohol is typically ethanol or isopropanol.

The pH of such liquid and other preparations of the invention is generally in an amount of about 4.5 to about 10. The pH can be controlled with acid (e.g., citric acid or benzoic acid) or base (e.g., sodium hydroxide) or buffered (with sodium citrate, benzoate, carbonate, or bicarbonate, disodium hydrogen phosphate, or sodium dihydrogen phosphate, for example).

In various embodiments, the aqueous oral composition (e.g., mouthrinse) contains a humectant. The humectant is generally a mixture of humectants, such as glycerin and sorbitol, and a polyhydric alcohol such as propylene glycol, butylene glycol, hexylene glycol, polyethylene glycol. The humectant content is in the range of about 5 to abut 40% and preferably about 10 to about 30%. Surfactants useful in the present embodiment include anionic, nonionic, and zwitterionic surfactants. The surfactant is present in the aqueous oral compositions of the present invention in an amount of about 0.01% to about 5%, preferably in an amount of about 0.5% to about 2.5%.

The term “confectionery composition” as used herein includes chewing gums, and orally soluble tablets, beads and lozenges. Saliva dissolves the lozenge or chewable gum product, and promotes prolonged contact with oral surfaces so that the delivery of the antibacterial agent and the anticalculus system in a lozenge tablet, bead or chewing gum form ensures that an adequate dosage of the active ingredients are delivered to the oral surface when the product is used.

In the present embodiment, the orally acceptable carrier is in the form of a lozenge, bead, tablet or chewing gum or other similar solid delivery system. Such delivery systems are well known to one of skill the art, and generally entail stirring the active antibacterial and anticalculus agents into a warm base with flavor, and non-cariogenic sweeteners.

The orally acceptable vehicle or carrier in a lozenge bead or tablet is a non-cariogenic, solid water-soluble polyhydric alcohol (polyol) such as mannitol, xylitol, sorbitol, malitol, hydrogenated starch hydrozylate, hydrogenated glucose, hydrogenated disaccharides or hydrogenated polysaccharides, in an amount of about 85 to about 95% of the total composition. Emulsifiers such as glycerin, and tableting lubricants, in minor amounts of about 0.1 to 5%, may be incorporated into the tablet, bead or lozenge formulation to facilitate the preparation of the tablet beads and lozenges. Suitable lubricants include vegetable oils such as coconut oil, magnesium stearate, aluminum stearate, talc, starch and CARBOWAX. Suitable noncariogenic gums include kappa carrageenan, carboxymethyl cellulose, hydroxyethyl cellulose and the like.

The lozenge, bead or tablet may optionally be coated with a coating material such as waxes, shellac, carboxymethyl cellulose, polyethylene/maleic anhydride copolymer or kappa-carrageenan to further increase the time it takes the tablet or lozenge to dissolve in the mouth. The uncoated tablet or lozenge is slow dissolving, providing a sustained release rate of active ingredients of about 3 to 5 minutes. Accordingly, the solid dose tablet, bead and lozenge compositions of this embodiment affords a relatively longer time period of contact of the teeth in the oral cavity with the anti-inflammatory active ingredients of the present invention.

The chewing gum of the present invention is preferably a sugarless chewing gum containing the antibacterial and anticalculus compounds. Chewing gum formulations typically contain, in addition to, a chewing gum base, one or more plasticizing agents, at least one sweetening agent and at least one flavoring agent.

Gum base materials suitable for use in the practice of this invention are well known in the art and include natural or synthetic gum bases or mixtures thereof. Representative natural gums or elastomers include chicle, natural rubber, jelutong, balata, guttapercha, lechi caspi, sorva, guttakay, crown gum, perillo, or mixtures thereof. Representative synthetic gums or elastomers include butadiene-styrene copolymers, polyisobutylene and isobutylene-isoprene copolymers. The gum base is incorporated in the chewing gum product at a concentration of about 10 to about 40% and preferably about 20 to about 35%.

Plasticizing/softening agents commonly used in chewing gum compositions are suitable for use in this invention, including gelatin, waxes and mixtures thereof in amounts of about 0.1 to about 5%. The sweetening agent ingredient used in the practice of this invention may be selected from a wide range of materials, and include the same artificial and polyol sweeteners used for the preparation of tablets, beads and lozenges. Polyol sweeteners such as sorbitol and malitol are present in the chewing gum composition of the present invention in amounts of about 40 to about 80% and preferably about 50 to about 75%. The artificial sweetener is present in the chewing gum composition of the present invention in amounts of about 0.1 to about 2% and preferably about 0.3 to about 1%.

In certain other desirable forms of this invention, the oral composition may be a dentifrice. As referred to herein, a “dentifrice” is a composition that is intended for cleaning an oral surface within the oral cavity. Such dentifrices include toothpowder, a dental tablet, toothpaste (dental cream), or gel. In a toothpaste dentifrice, the orally acceptable carrier may comprise water and humectant typically in an amount of about 10% to about 80% of the oral composition.

In various embodiments of the present invention, glycerin, propylene glycol, sorbitol, polypropylene glycol and/or polyethylene glycol (e.g., 400-600) are suitable humectants/carriers. Also advantageous are liquid mixtures of water, glycerin and sorbitol. In certain embodiments where the carrier is a clear gel and where the refractive index is an important consideration, the composition comprises about 3 to about 30% of water, 0 to about 70% of glycerin and about 20-80% of sorbitol.

In various embodiments, such as for toothpastes, creams and gels, the oral composition contains a natural or synthetic thickener or gelling agent, which other than silica thickeners, include natural and synthetic gums and colloids. Such suitable thickeners include naturally occurring polymers such as carrageenans, xanthan gum, synthetic thickener such as polyglycols of varying molecular weights sold under the tradename POLYOX and cellulose polymers such as hydroxyethyl cellulose and hydroxpropyl cellulose. Other inorganic thickeners include natural and synthetic clays such as hectorite clays, lithium magnesium silicate (laponite) and magnesium aluminum silicate. Other suitable thickeners are synthetic hectorite, synthetic colloidal magnesium alkali metal silicate complex clay available for example as Laponite (e.g., CP, SP 2002, or D) marketed by Laporte Industries Limited. Laponite D analysis shows, approximately, 58.00% SiO₂, 25.40% MgO, 3.05% Na₂O, 0.98% Li₂O, and some water and trace metals, and has a true specific gravity of 2.53 and an apparent bulk density (g/mL at 8% moisture) of 1.0. In certain embodiments, the thickening agent is present in the dentifrice composition in amounts of about 0.1 to about 10%, preferably about 0.5 to about 5.0%.

Other suitable thickeners include Irish moss, gum tragacanth, starch, polyvinylpyrrolidone, hydroxyethyl propyl cellulose, hydroxybutyl methyl cellulose, hydroxypropyl methyl cellulose, hydroxyethyl cellulose, sodium carboxymethyl cellulose, and colloidal silica such as finely ground Syloid (e.g. 244).

Various embodiments of the present invention also comprise a surface active agent, which may function as a surfactant, emulsifier, and/or foam modulator. Surface active agents generally achieve increased prophylactic action, by thoroughly dispersing the active ingredients throughout the oral cavity. Suitable emulsifying agents are those which are reasonably stable and foam throughout a wide pH range, including non-soap anionic, nonionic, zwitterionic and amphoteric organic synthetic detergents. Further, surface active ingredients preferably render the instant compositions more cosmetically acceptable. The organic surface-active material is preferably anionic, nonionic or ampholytic in nature, and preferably a detersive material which imparts to the composition detersive and foaming properties. In certain embodiments, one or more surfactants are present in the oral composition of the present invention in an amount of about 0.1% to about 5% preferably in an amount of about 0.6% to about 2.0%.

Nonionic surfactants useful in the compositions of the present invention include compounds produced by the condensation of alkylene oxides (especially ethylene oxide) with an organic hydrophobic compound, which may be aliphatic or alkylaromatic in nature. One group of surfactants is known as “ethoxamers”—they are condensation products of ethylene oxide with fatty acids, fatty alcohols, fatty amides, polyhydric alcohols, (e.g., sorbitan monostearate) and the like. “Polysorbates” is the name given to a class of nonionic surfactants prepared by ethoxylating the free hydroxyls of sorbitan-fatty acid esters. They are commercially available, for example as the TWEEEN® surfactants of ICI. Non-limiting examples include Polysorbate 20 (polyoxyethylene 20 sorbitan monolaurate, TWEEN® 20) and Polysorbate 80 (polyoxyethylene 20 sorbitan mono-oleate, TWEEN® 80). Preferred polysorbates include those with about 20 to 60 moles of ethylene oxide per mole of sorbitan ester.

Other suitable nonionic surfactants include poly(oxyethylene)-poly(oxypropylene) block copolymers, especially triblock polymers of this type with two blocks of poly(oxyethylene) and one block of poly(oxypropylene). Such copolymers are known commercially by the non-proprietary name of poloxamers, the name being used in conjunction with a numeric suffix to designate the individual identification of each copolymer. Poloxamers may have varying contents of ethylene oxide and propylene oxide, leading to a wide range of chemical structures and molecular weights. One preferred poloxamer is Poloxamer 407. It is widely available, for example under the tradename PLURONIC® F127 of BASF Corporation.

Other non-limiting examples of suitable nonionic surfactants include products derived from the condensation of ethylene oxide with the reaction product of propylene oxide and ethylene diamine, long chain tertiary amine oxides, long chain tertiary phosphine oxides, long chain dialkyl sulfoxides and the like.

Other surfactants useful in various embodiments of the present invention include zwitterionic synthetic surfactants. Certain of these can be broadly described as derivatives of aliphatic quaternary ammonium, phosphonium, and sulfonium compounds, in which the aliphatic radicals can be straight chain or branched, and where one of the aliphatic substituents contains from 8 to 18 carbon atoms and one contains an anionic water-solubilizing group, e.g., carboxy, sulfonate, sulfate, phosphate or phosphonate. One example of a suitable zwitterionic surfactant is 4-(N,N-di(2-hydroxyethyl)-N-octadecylammonio)-butane-1-carboxylate.

Other suitable zwitterionic surfactants include betaine surfactants, such as those disclosed in U.S. Pat. No. 5,180,577. Typical alkyldimethyl betaines include decyl betaine 2-(N-decyl-N,N-dimethylammonio) acetate, cocobetaine, myristyl betaine, palmityl betaine, lauryl betaine, cetyl betaine, stearyl betaine, and the like. The amidobetaines are exemplified by cocoamidoethyl betaine, cocoamidopropyl betaine, lauramidopropyl betaine and the like. Particularly useful betaine surfactants include cocoamidopropyl betaine and lauramido propyl betaine.

Suitable examples of anionic surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine which are preferably substantially free from soap or similar higher fatty acid material.

In various embodiments of the present invention, where the carrier of the oral care composition is solid or a paste, the oral composition preferably comprises a dentally acceptable abrasive material, which may serve to either polish the tooth enamel or provide a whitening effect. In the preparation of a dentifrice composition, abrasives which may be used in the practice of the present invention include silica abrasives such as precipitated silicas having a mean particle size of up to about 20 microns, such as ZEODENT® 115, marketed by J. M. Huber. One useful abrasive is marketed under the trade designation ZEODENT® 105 by J. M Huber Co, which has a low abrasiveness to tooth enamel, and is a precipitated silica that is about 7 to about 10 microns in diameter, has a BET surface area of 390 m²/g of silica, and an oil absorption of less than 70 cm³/100 g of silica. Other useful dentifrice abrasives include sodium metaphosphate, potassium metaphosphate, tricalcium phosphate, dihydrated dicalcium phosphate, aluminum silicate, calcined alumina, bentonite or other siliceous materials, or combinations thereof.

In other embodiments of the present invention, useful abrasive materials for preparing dentifrice compositions include silica gels and precipitated amorphous silica having an oil absorption value of less than 100 cm³/100 g silica and preferably in an amount of about 45 cm³/100 g to less than about 70 cm³/100 silica. Oil absorption values are measured using the ASTA Rub-Out Method D281. These silicas are colloidal particles having an average particle size ranging in an amount of about 3 microns to about 12 microns, and more preferably about 5 to about 10 microns and a pH range of about 4 to 10, preferably about 6 to 9, when measured as a 5% slurry.

Further suitable abrasives useful with various embodiments of the present invention are low oil of absorption silica abrasives such as those marketed under the trade designation SYLODENT® XWA or SYLODENT® 783 by Davison Chemical Division of W. R. Grace & Co., Baltimore, Md., United States of America. SYLODENT® XWA 650, a silica hydrogel composed of particles of colloidal silica having a water content of 29%, averaging about 7 to about 10 microns in diameter, and an oil absorption of less than 70 cm³/100 g of silica is a preferred example of a low oil absorption silica abrasive useful in the practice of the present invention. The abrasive is present in the dentifrice composition of the present invention at a concentration of about 10 to about 40% and preferably about 15 to about 30%.

Other suitable polishing materials include the particulate thermosetting resins, such as melamine, phenolic, and urea-formaldehydes, and cross-linked polyepoxides and polyesters. Preferred polishing materials include crystalline silica having particle sizes of up to about 5 microns, a mean particle size of up to about 1.1 microns, and a surface area of up to about 50,000 cm²/g, silica gel or colloidal silica, and complex amorphous alkali metal aluminosilicate.

In embodiments where the dentifrice is a clear or transparent gel, a polishing agent of colloidal silica, such as those sold under the trademark SYLOID® or under the trademark SANTOCEL® alkali metal almuino-silicate complexes are particularly useful, since they have refractive indices close to the refractive indices of gelling agent-liquid (including water and/or humectant) systems commonly used in dentifrices.

Many of the so-called “water-insoluble” polishing materials are anionic in character and also include small amounts of soluble material. Thus, insoluble sodium metaphosphate, known as Madrell's salt and Kurrol's salt are examples of suitable polishing materials. These metaphosphate salts exhibit only a minute solubility in water, and therefore are commonly referred to as insoluble metaphosphates (IMP). Such IMPs generally contain a minor amount, usually a few percent (e.g., <4%), of soluble phosphate material as impurities. Some of these impurities can be removed by pre-washing the material. The insoluble alkali metal metaphosphate is typically employed in powder form of a particle size such that no more than 1% of the material is larger than 37 microns.

In certain embodiments, the abrasives may also include whiteness-imparting abrasive particles which include for example, a metal oxide. The metal oxide can comprise any metal oxide that provides a white color, such as, for example, titanium oxide, aluminum oxide, tin oxide, calcium oxide, magnesium oxide, barium oxide, or a combination thereof. Certain whiteness imparting abrasives are also pearlescent particles, which comprise a single mineral or chemical species, such as, for example a silicate such as mica, or bismuth oxychloride. By “mica” it is meant any one of a group of hydrous aluminum silicate minerals with platy morphology and perfect basal (micaceous) cleavage. Mica can be, for example, sheet mica, scrap mica, or flake mica, as exemplified by muscovite, biotite or phlogopite type micas. In some embodiments, the pearlescent particles can comprise a complex comprising more than one mineral or chemical species, such as, for example, mica coated with a metal oxide such as titanium oxide.

In embodiments where the dentifrice is in a solid or paste form, the abrasive material is generally present at about 10% to about 99% of the oral composition. In certain embodiments, the polishing material is present in an amount of about 10% to about 75% in toothpaste, and of about 70% to about 99% in toothpowder.

In various embodiments of the present invention, water is also present in the oral composition, as referred to above. Water employed in the preparation of commercially suitable toothpastes, gels, and mouthwashes should preferably be deionized and free of organic impurities. Water generally comprises about 10% to 50%, preferably about 20% to 40%, of the toothpaste compositions herein. The water is free water which is added, plus that which is introduced with other materials for example, such as that added with sorbitol.

In various embodiments, the oral care composition of the present invention contains a flavoring agent. Flavoring agents which are used in the practice of the present invention include essential oils as well as various flavoring aldehydes, esters, alcohols, and similar materials. Any suitable flavoring or sweetening material may also be employed. Examples of suitable flavoring constituents are flavoring oils, e.g. oil of spearmint, peppermint, wintergreen, sassafras, clove, sage, eucalyptus, marjoram, cinnamon, lemon, lime, orange, grapefruit, and methyl salicylate. Also useful are such chemicals as menthol, carvone, and anethole. Suitable sweetening agents include sucrose, lactose, maltose, sorbitol, xylitol, sodium cyclamate, perillartine, AMP (aspartyl phenyl alanine, methyl ester), saccharine and the like. The flavor and sweetening agents may each or together be incorporated into the oral composition at a concentration of about 0.001 to about 5% and preferably about 0.5 to about 2.0%.

In certain embodiments of the present invention, the oral composition may be in the form of a non-abrasive medicament gel. By “medicament” it is meant that the gel is provided for medicinal treatment or therapy for purposes of healing or ameliorating the detrimental condition or disease. The present embodiment is not intended to limit those compositions which are useful as medicaments, as the previously described forms of the oral compositions are also suitable as medicaments. However, the oral compositions provided in a non-abrasive gel or ointment form are particularly useful for localized treatment and can be used in conjunction with wound dressings, gauze, films, and the like. Such gels may include both aqueous and non-aqueous gels, and include those formulations previously described above.

Certain aqueous gels are particularly suitable for application to the gingival sulcus or margin, and for subgingival application. Such aqueous gels generally comprise a thickener in an amount of about 0.1 to about 20%, a humectant in an amount of about 10 to about 55%, a flavoring agent in an amount of about 0.01 to about 2%, a sweetening agent in an amount of about 0.1 to about 3%, optionally a coloring agent (in an amount of about 0.01 to about 0.5%), and the balance water. Gels may comprise a polymer carrier comprising polymers selected from the group of polylactic acid, polyglycolic acid, polylactyl-co-glycolic acid, polyaminoacids such as polyaspartame, chitosan, collagen, polyalbumin, gelatin, and hydrolyzed animal protein, polyvinyl pyrrolidone, xanthan and other water soluble gums, polyanhydride, and polyorthoesters. In certain embodiments, the gel comprises polymers and copolymers of polylactic acid, polyglycolic acid, and poly lactyl-co-glycolic acid. In other embodiments, the gel comprises copolymers of lactide and gycolide monomers, where lactide comprises about 15 to about 85%, most preferably in an amount of about 35 to about 65%, and glycolide monomeric species comprise about 15% to about 85%, preferably about 35 to about 65% on a molar basis.

It has long been known that dental prophylaxis is promoted in pets, and especially dogs, cats, and horses, by the scraping of relatively hard surfaces against the pet's teeth by chewing (for example, bone chewing). Incidental contact with active ingredients associated with the animal products further promotes dental health in animals. Thus, in certain embodiments, the active ingredients of the present invention can be incorporated in animal food products, supplemental food products (e.g., pet treats), chew articles, and the like.

Chew articles or toys can be formed in a variety of designs and sizes, as known to those of skill in the art, and preferably provide some level of physical interaction with the tooth and gum surface, promoting gingival stimulation and/or sub-gingival particle release. Examples of such toys can be bones, balls, and ropes. Further, it is preferred that the chew toys are capable of carrying active ingredients, either through an internal reservoir, by impregnation into the material, or coating onto a surface of the toy, for example. Chew articles of the present embodiment are preferably formed of a non-toxic edible material, including by way of example, rawhide or polymers such as polyester or polyisoprene.

Food products and supplements for animals are well known in the art and are preferably made with any suitable dough. Food supplement dough generally comprises at least one of flour, meal, fat, water, and optionally particulate proteinaceous particles (for texturization) and flavor. For instance, when the desired product is a biscuit, conventional dough can be used, optionally containing discrete particles of meat and/or meat by-products or farinaceous material. Examples of suitable dough for the production of hard and soft (including humectant for water control) animal biscuits are disclosed in U.S. Pat. Nos. 5,405,836; 5,000,943; 4,454,163; 4,454,164, the contents of each of which are incorporated herein by reference. Such compositions are preferably baked. The active ingredient may be added with the flavor, included in an interior reservoir with a soft center, or coated onto the surface of a baked food supplement by dipping or spraying. Any other suitable means known to one of skill in the art for delivering active ingredients to animals are also contemplated by the present invention.

The compositions used in accordance with the present invention optionally comprise an optional active material aside from the anti-inflammatory active ingredient consisting essentially of an extract of magnolia, which is operable for the prevention or treatment of a condition or disorder of hard or soft tissue of the oral cavity, the prevention or treatment of a physiological disorder or condition, or to provide a cosmetic benefit. In such embodiments, the one or more additional active ingredients do not inhibit the efficacy of the anti-inflammatory ingredients previously described and generally such additional ingredients are not known to have anti-inflammatory properties.

Additional actives agents among those useful for the composition described herein are disclosed in, e.g., U.S. Pat. No. 6,290,933 and U.S. Pat. No. 6,685,921, the contents of each of which are incorporated herein by reference.

The oral composition of the present invention may contain an anticaries agent, such as a fluoride ion source or a fluorine-providing component. In various embodiments, the fluoride based anticaries agent in present in an amount sufficient to supply about 25 ppm to 5,000 ppm of fluoride ions. Useful anticaries agents include inorganic fluoride salts, such as soluble alkali metal salts. For example, preferred fluoride sources useful in the composition are sodium fluoride; potassium fluoride; sodium fluorosilicate; ammonium fluoro silicate; amine fluorides; including olaflur (N′-octadecyltrimethylendiamine-N,N,N′-tris(2-ethanol)-dihydrofluoride); as well as tin fluorides, such as stannous fluoride and stannous chloride.

In various embodiments, the oral compositions of the present invention comprise antitartar agents to prevent and/or minimize calculus formation. One or more of such agents can be present.

Suitable anticalculus agents include without limitation: phosphates and polyphosphates. Phosphate and polyphosphate salts are generally employed in the form of their wholly or partially neutralized water soluble cationic species (e.g., potassium, sodium or ammonium salts, and any mixtures thereof). Thus, useful inorganic phosphate and polyphosphate salts illustratively include monovalent cations with monobasic, dibasic and tribasic phosphates; tripolyphosphate and tetrapolyphosphate; mono-, di-, tri- and tetra-pyrophosphates; and cyclophosphates (also generally known in the art as “metaphosphates”). Useful monovalent cations of such phosphate salts include hydrogen, monovalent metals including alkali metals, and ammonium, for example.

Additionally, various embodiments of the present invention include an anticalculus system that further comprises a synthetic anionic linear polycarboxylate polymer. The anionic linear polycarboxylate is generally synthesized by using an olefinically or ethylenically unsaturated carboxylic acid that contains an activated carbon-to-carbon olefinic double bond and at least one carboxyl group. The acid contains an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrilacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other olefinic monomers copolymerizable with such carboxylic monomers include vinyl acetate, vinyl chloride, dimethyl maleate and the like. The synthetic anionic linear polymeric polycarboxylate component is mainly a hydrocarbon with optional halogen and O-containing substituents and linkages as present in for example ester, ether and OH groups. The copolymers preferably contain sufficient carboxylic salt groups for water-solubility. The terms “synthetic” and “linear” do not include known thickening or gelling agents comprising carboxymethylcellulose and other derivatives of cellulose and natural gums, nor Carbopols having reduced solubility due to cross-linkages.

Various optional oral care actives may be included in the oral composition of the present invention including those described above, such as antibacterial agents (such as, botanical extracts or galenical active compounds), antiplaque agents, anti-adhesion agents (that prevent adhesion of plaque to an enamel surface, such as, N^{α}-acyl amino acid alkyl esters, including N^{α}-lauroyl-L-arginine ethyl ester hydrochloride), anti-oxidants (such as, Vitamin E or coenzyme Q10), anticaries agents, densensitizing agents (such as, potassium citrate, potassium tartrate, potassium chloride, potassium sulfate and potassium nitrate), whitening agents (such as, urea peroxide, sodium percarbonate, sodium perborate and polyvinylpyrrolidone-H₂O₂); compatible enzymes; tartar control agents, periodontal actives, chlorophyll compounds, nutrients (such as, vitamins, minerals, and amino acids, lipotropics, fish oil, coenzymes and the like) abrasives, breath freshening/malodor control agents (such, as zinc salts such as zinc gluconate, zinc citrate, zinc chlorite, and α-ionone), and salivary stimulants (such as, such as citric, lactic, malic, succinic, ascorbic, adipic, fumaric and tartaric acids); and any other suitable ingredients for oral care known to one of skill in the art. These additives, when present, are incorporated in the oral composition in amounts that do not substantially adversely affect the properties and characteristics desired, generally from concentrations of about 0.001 to about 10%.

Various other materials may be incorporated in oral compositions of this invention including preservatives, such as sodium benzoate, and silicones, for example. These adjuvants, when present, are incorporated in the compositions in amounts which do not substantially adversely affect the properties and characteristics desired.

### EXAMPLE I

A dentifrice composition having the ingredients listed in Table I is prepared by the following method. The magnolia extract obtained by isolation with HFA-13A has approximately 15% by weight honokiol and 37% by weight magnolol.

Sodium saccharin, sodium fluoride, and any other salts are dispersed in water and mixed in a conventional mixer under agitation. The humectants e.g., glycerin and sorbitol, are added to the water mixture under agitation. Then organic thickeners, such as carrageenan and CMC, as well as any polymers are added. The resultant mixture is agitated until a homogeneous gel phase is formed. The mixture is then transferred to a high-speed vacuum mixer; where the SYLODENT® XWA 650 and SYLODENT® 783 abrasive and silica thickener ZEODENT® 165 are added. The mixture is then mixed at high speed for from 5 to 30 minutes, under vacuum of in an amount of about 20 to 50 mm of Hg, preferably about 30 mm Hg. The flavor oil is weighed out and magnolia is then added to the flavor oil. The flavor oil and magnolia mixture is added to the mixture. Lastly, surfactants, such as sodium lauryl sulfate (SLS) are charged into the mixer. The resultant product is a homogeneous, semi-solid, extrudable paste or gel product.

TABLE I

| Ingredient | Wt. % |
|---|---|
| Magnolia Cortex Extract | 0.3 |
| Sorbitol (70% in H₂O) | 26.7 |
| Glycerin | 12.0 |
| Sodium fluoride | 0.3 |
| Sodium saccharin | 0.5 |
| Sodium hydroxide (50% in H₂O) | 2.0 |
| CMC 2000S | 0.8 |
| Carrageenan (LB 9505) | 0.4 |
| Sylodent 783 | 11.0 |
| Sylodent XWA 650 | 10.0 |
| Zeodent 165 | 3.5 |
| Sodium lauryl sulfate (30% conc.) | 4.0 |
| TiO₂ coated Mica | 0.1 |
| Flavor (89-332) | 1.0 |
| Blue Color Solution | 0.05 |
| Water | Q.S. |

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

We claim:

1. A method of treating a mammalian subject having oral tissue inflammation, the method comprising contacting the tissue with an oral composition comprising an anti-inflammatory active ingredient consisting essentially of an extract of magnolia and an orally acceptable carrier, wherein the oral composition reduces inflammation of the oral tissue by reducing one or more mediators of inflammation.

2. The method according to claim 1, wherein during the contacting, the oral composition further provides an analgesic effect on the tissue, thereby reducing sensations of pain and sensitivity of the oral tissue in the mammalian subject.

3. The method according to claim 1, where the concentration of the magnolia extract is less than 0.5%.

4. The method according to claim 1, where the concentration of the magnolia extract is less than 0.3%.

5. The method according to claim 1, wherein the magnolia extract comprises an active compound selected from the group consisting of: magnolol, honokiol, tetrahydromagnolol, tetrahydrohonokiol, and mixtures thereof.

6. The method according to claim 1, wherein the extract of magnolia comprises about 2% to about 95% of an active compound selected from the group consisting of: magnolol, honokiol, or mixtures thereof.

7. The method according to claim 1, wherein the orally acceptable carrier comprises one or more oral active ingredients selected from the group consisting of: anti-tartar agents, antibacterial agents, anticaries agents, whitening agents, densensitizing agents, vitamins, compatible enzymes, breath freshening agents, malodor preventing agents, and combinations thereof.

8. The method according to claim 1, wherein the orally acceptable carrier comprises one or more components selected from the group consisting of: viscosity modifiers, diluents, surface active agents, pH modifying agents, abrasives, humectants, mouth feel agents, sweetening agents, flavor agents, colorants, preservatives, and combinations thereof.

9. The method according to claim 1, wherein the oral tissue inflammation in the mammalian subject is associated with chronic pathogenic infection.

10. The method according to claim 1, wherein the oral tissue inflammation is associated with a condition selected from the group consisting of: tooth loss, oral surgery, endodontic pathoses, stomatitis, alveolar bone resorption, lesions, gingivitis, periodontitis, tobacco induced disease, and combinations thereof.

11. The method according to claim 1, wherein one or more of the mediators of inflammation is a cytokine.

12. The method according to claim 1, wherein the one or more of the mediators of inflammation is a prostaglandin.

13. The method according to claim 1, wherein the contacting is repeated for a plurality of days to reduce inflammation.

14. The method according to claim 1, wherein the oral care composition is in a form selected from the group consisting of: a mouthrinse, a dentifrice, an animal product, a medicament gel, and a dentifrice.

15. A method of reducing oral tissue inflammation in a mammalian subject, the method comprising contacting the tissue with an oral composition comprising a non-irritating amount of an anti-inflammatory active ingredient consisting essentially of an extract of magnolia, and an orally acceptable carrier, wherein the oral composition does not irritate the oral tissue and further reduces inflammation of the oral tissue by reducing one or more mediators of inflammation.

16. The method according to claim 15, wherein after the contacting, the non-irritating amount of the oral composition relates to one or more active ingredients of the magnolia extract present near the oral tissue at a concentration of less than 20 μg/mL.

17. The method according to claim 15, wherein after the contacting, the non-irritating amount of the oral composition relates to one or more active ingredients of the magnolia extract present near the oral tissue at a concentration of less than 10 μg/mL.

18. The method according to claim 15, wherein during the contacting, the oral composition further provides an analgesic effect on the tissue, thereby reducing sensations of pain and sensitivity of the oral tissue in the mammalian subject.

19. The method according to claim 15, where the concentration of the magnolia extract in the oral composition is less than or equal to 0.3%.

20. The method according to claim 15, wherein the extract of magnolia comprises about 2% to about 95% of an active compound selected from the group consisting of: magnolol, honokiol, or mixtures thereof.

21. The method according to claim 15, wherein the orally acceptable carrier comprises one or more oral active ingredients selected from the group consisting of: anti-tartar agents, antibacterial agents, anticaries agents, whitening agents, densensitizing agents, vitamins, compatible enzymes, breath freshening agents, malodor preventing agents, and combinations thereof.

22. The method according to claim 15, wherein the orally acceptable carrier comprises one or more components selected from the group consisting of: viscosity modifiers, diluents, surface active agents, pH modifying agents, abrasives, humectants, mouth feel agents, sweetening agents, flavor agents, colorants, preservatives, and combinations thereof.

23. The method according to claim 15, wherein one or more of the mediators of inflammation is a cytokine.

24. A method for treating a mammalian subject having oral tissue inflammation, the method comprising contacting the inflamed oral tissue with an oral composition comprising an orally acceptable carrier and an anti-inflammatory active ingredient consisting essentially of an extract of magnolia, wherein the oral composition reduces inflammation by reducing one or more mediators of inflammation, and wherein the orally acceptable carrier comprises one or more oral active ingredients selected from the group consisting of: anti-tartar agents, antibacterial agents, anticaries agents, whitening agents, densensitizing agents, vitamins, compatible enzymes, breath freshening agents, malodor preventing agents, and combinations thereof.

25. The method according to claim 24, wherein the orally acceptable carrier further comprises one or more components selected from the group consisting of: viscosity modifiers, diluents, surface active agents, pH modifying agents, abrasives, humectants, mouth feel agents, sweetening agents, flavor agents, colorants, preservatives, and combinations thereof.
